(19) United States
(12) Patent Application Publication
(10) Pub. No.: US 2021/0093205 A1
(43) Pub. Date: Apr. 1, 2021

(54) REAL-TIME SENSING MEASUREMENT AND INTELLIGENT CONTROL TECHNOLOGY APPARATUS FOR A RESPIRATORY VOLUME OF A TESTED PERSON

(71) Applicant: Chinese Research Academy of Environmental Sciences, Beijing (CN)

(72) Inventors: Xiuge ZHAO, Beijing (CN); Danlu WANG, Beijing (CN); Zhenglei LI, Beijing (CN); Limin LEI, Beijing (CN); Ju HUANG, Beijing (CN); Puqiu ZHAO, Beijing (CN); Feiyang QIAO, Beijing (CN)

(73) Assignee: Chinese Research Academy of Environmental Sciences, Beijing (CN)

(21) Appl. No.: 17/015,097

(22) Filed: Sep. 9, 2020

(30) Foreign Application Priority Data
Sep. 29, 2019 (CN) 201910933187.3

(51) Int. Cl. A61B 5/0205; A61B 5/00; A41D 13/11; A62B 18/08; A62B 18/02; A62B 18/10

(52) U.S. Cl. CPC A61B 5/02055; A61B 5/6803; A41D 13/11; A61B 5/02438; A62B 18/08; A62B 18/02; A62B 18/10; A61B 5/4836

(57) ABSTRACT

A real-time sensing measurement apparatus for a respiratory volume of a tested person includes a controller, a pulse measuring device, a temperature sensor, a flow rate sensor and a storage module. The controller receives an electric signal transmitted by the pulse measuring device, the temperature sensor and the flow rate sensor. The controller, the pulse measuring device, the temperature sensor, the flow rate sensor and the storage module communicate through electric signals. The pulse measuring device, the temperature sensor and the flow rate sensor monitor the respiratory volume of the tested person in real time to obtain test data by the storage module. The different states of the user can be measured in real time by the pulse measuring device, the temperature sensor and the flow rate sensor, and measurement results are rectified in various aspects.

### CROSS REFERENCE TO THE RELATED APPLICATIONS

This application is based upon and claims priority to Chinese Patent Application No. 201910933187.3, filed on Sep. 29, 2019, the entire contents of which are incorporated herein by reference.

### TECHNICAL FIELD

The present invention relates to the technical field of measuring respiratory volumes, more particularly, the measuring of respiratory volumes using real-time sensing and intelligent control technology apparatus.

### BACKGROUND

A tested person's respiratory volumes refer to the volume of air that the tested person inhales per unit time, which can be separately categorized as short-term respiratory volumes and long-term respiratory volumes. The value of the short-term respiratory volumes is calculated according to the volume of air inhaled per minute or hour (L/min or m³/h). According to the level of activity intensity, which the tested person engages during testing, the short-term respiratory volumes in a measurement of the volume of air that the tested person inhales during resting, sitting, low-intensity exercising, moderate-intensity exercising, and high-intensity exercising. The value of the long-term respiratory volumes is calculated according to the volume of air inhaled per day (m³/d). The value of respiratory volumes is affected by different factors, e.g., the age and gender of the tested person, and the intensity of activities which the tested person engages during testing.

Air pollution greatly harms human health. Whether based on direct measurement or model estimation method, an accurate measurement of the respiratory volumes is needed for health risk assessment of human air exposure, which has become a key factor to determine the accuracy of assessment results. The Environmental Protection Law and the Health); China Action (019-2030) stipulate that “the system of environmental and health monitoring, investigation and risk assessment must be established and developed”. In order to implement the relevant laws and regulations and the spirit of documents, the environmental and health monitoring, investigation and risk assessment have been in full swing in China. At present, the recommended respiratory rate of Chinese people is obtained according to the foreign estimation model. Due to the differences in race, social and economic conditions and living habits, the estimation results cannot truly reflect the actual situation of the Chinese population. Moreover, there are great differences in respiratory capacity under different exercise states. The current recommended values of respiratory volume cannot reflect the dynamic changes in respiratory volume. Up to now, there are just a few studies on accurate measurement and accurate estimation of a person's respiratory volumes under different conditions. Therefore, it is necessary to establish an accurate estimation method and develop an automatic control system of respiratory volume based on the behavior pattern of the Chinese population, so as to provide effective solutions for accurately monitoring the important parameters of human air-exposed behavior patterns in China.

The existing respiratory volume measuring devices only measure tested persons Who are in hospitals or special testing facilities. These devices are cumbersome as they cannot be easily moved. This limits the population that can be tested as people frequently move around and are not always available to go to a specialized testing facility or hospitals for regular measurements. That makes it near impossible to obtain real-time respiratory measurement data from mobilized population. In addition, the value of the respiratory volumes of the same tested person under different conditions are different, the existing devices can only measure the respiratory volumes of a person under stable conditions, and not able to obtain real-time respirator)/volume measurement data according to the different states of activity, which the person engages. Because the measuring device cannot adjust to the tested person's movement state in real-time, it frequently produces erroneous data, and undermines the accuracy of the measurement results. Because people are not measured in real time, the timeliness of the measured data cannot be guaranteed.

The present invention overcomes the shortcomings of the prior art, by providing an apparatus for real-time sensing and measuring, controlled by intelligent control technology, the respiratory volumes of a tested person. The apparatus measures respiratory volumes in real time and automatically adjust its opening degree, which solves the problems raised in the prior art.

### SUMMARY

The present invention provides the following technical solution: a real-time sensing measurement apparatus for detecting and evaluating the respiratory volumes of a tested person; the apparatus includes a controller, a pulse measuring device, a temperature sensor, a flow rate sensor and a storage module. The controller receives electric signals transmitted by the pulse measuring device, the temperature sensor and the flow rate sensor. The controller, the pulse measuring device, the temperature sensor, the flow rate sensor and the storage module communicate with each other through the electric signals. The pulse measuring device, the temperature sensor and the flow rate sensor monitor the respiratory volumes of the tested person in real time. The test data, which includes the data generated by the pulse measuring device, the temperature sensor, the flow rate sensor monitor, is stored in the storage module.

Preferably, the pulse measuring device is configured to measure a heart rate and a pulse rate, the temperature sensor is configured to sense an inhaled air temperature of the tested person and an exhaled air temperature of the tested person. The flow rate sensor is configured to sense an inhaled air flow rate of the tested person and an exhaled air flow rate of the tested person. The temperature sensor communicates with the flow rate sensor.

Preferably, the person to be tested using the measurement apparatus measures his respiratory volumes through the pulse measuring device, the temperature sensor and the flow rate sensor, and then data is transferred to the storage module. A processor in the storage module classifies the acquired data. The classified data includes a value for minutely respiratory volumes, hourly respiratory volumes, and daily respiratory volumes. The value for minutely respiratory volumes, the hourly respiratory volumes and the daily respiratory volumes are all stored in the storage module. A dynamic estimation model of the tested person's respiratory volumes is built by analyzing the relationship between his/her minutely respiratory volumes, hourly respiratory volumes, daily respiratory volumes, pulse, temperature, and inhalation/exhaled air flow rate.

The intelligent control technology apparatus for measuring the respiratory volumes of a tested person includes a mask, a flow rate sensor, a flow rate automatic control valve, and the controller. The flow rate sensor, the flow rate automatic valve, and the controller are all connected to the mask. The flow rate sensor obtains the respiratory volumes inhaled and exhaled by the tested person. Air is exhaled and inhaled through the flow rate automatic control valve, and the respiratory volumes of each exhalation and inhalation is measured by the flow rate sensor. If the respiratory volumes of each exhalation and inhalation is higher than a pre-determined threshold value which is stored in the controller and is set according to a maximum respiratory volume in big data, the flow rate sensor will send the respiratory volume of each exhalation and inhalation to the flow rate automatic control valve. The respiratory volumes of each exhalation and inhalation will be judged by the controller. If the respiratory volumes of each exhalation and inhalation is lower or higher than a normal threshold value, the controller will activate an actuator to control sensitive elements inside the flow rate automatic control valve and control an opening size of the flow rate automatic control valve. After adjustment, an electric signal will be transmitted to the controller by the flow rate automatic control valve.

Preferably, the intelligent control technology apparatus has the option to operate in three different modes, consisting of an exercising state, a weight-bearing state and a walking state. The exercising state, the weight-bearing state and the walking state are respectively set with different thresholds, and the thresholds of the exercising state; the weight-bearing state and the walking state include a minimum opening degree, a normal opening degree and a maximum opening degree. Each mode corresponds to three different opening degrees of a breather valve. The opening degree of the breather valve is minimum when air velocity (±20%) is satisfied with P5 (5th percentile) of breath required for each state; the opening degree of the breather valve is normal when air velocity (±20%) is satisfied with an average amount of breath required for each state; the opening degree of the breather valve is maximum when air velocity (±20%) is satisfied with P95 (95th percentile) of breath required for each state. The flow rate sensor, the flow rate automatic control valve and the controller open the maximum opening degree when the system detects a large respiratory volumes of the tested person; the flow rate sensor, the flow rate automatic control valve and the controller open the normal opening degree when a normal respiratory volumes is detected; and the flow rate sensor, the flow rate automatic control valve and the controller open the minimum opening degree when a small respiratory volumes is detected.

The present invention has the following advantages:

1. The real-time sensing measurement and intelligent control technology apparatus can be worn on the face of the user by the mask, to which the pulse measuring device, the temperature sensor and the flow rate sensor are connected, so that the pulse measuring device, the temperature sensor and the flow rate sensor can measure the respiratory volumes of the user in real time, and store the data in the storage module. The different states of the user, e.g., exercising state, weight-bearing state, or walking state, can be measured in real time by the pulse measuring device, the temperature sensor and the flow rate sensor, and measurement results are rectified in various aspects to ensure that the measured respiratory volumes of the user is more accurate. Moreover, real-time data are stored at the same time, which facilitates the user to know his daily, hourly or minutely respiratory volumes, and conveniently exports the data to professionals for monitoring and analyzing. In this way, the respiratory volume can be measured without going through specialized institutions and hospitals. On the one hand, the present invention reduces the volume of the measuring apparatus, on the other hand, it increases the accuracy of the measurement.

2. The real-time sensing measurement and intelligent control technology apparatus can set a threshold value according to the respiratory volumes under different conditions, e.g., normal and abnormal conditions, by means of the controller, the flow rate sensor and a flow rate automatic control valve, so that the apparatus can set different thresholds according to different users, which is more personalized for each user. The measurement data and measurement effect are more accurate comparing to the data collected by devices known in the prior art as the embodiment of the present invention can adapt to different states, and automatically adjust according to the user's respiratory volumes of exhalation and inhalation.

### BRIEF DESCRIPTION OF THE DRAWINGS

FIG. 1 shows a flow chart of a respiratory volumes measurement in one embodiment of the present invention;

FIG. 2 shows a flow chart of a respiratory volumes control in one embodiment of the present invention;

FIG. 3 is a schematic diagram of a flow rate automatic control valve in one embodiment of the present invention;

FIG. 4 is a schematic diagram of an intelligent control technology apparatus measurement in one embodiment of the present invention.

In FIG. 4: 1. Mask; 2. Flow rate sensor; 3. Flow rate automatic control valve; 4. Controller.

### DETAILED DESCRIPTION OF THE EMBODIMENTS

Hereinafter, the technical solution in the embodiment of the present invention will be described clearly and completely in combination with the drawings. Obviously, the described embodiment is only a part of the embodiment of the present invention, rather than all the embodiments. Based on the embodiment of the present invention, all other embodiments obtained by ordinary skill in the art without creative labor belong to the scope of protection of the present invention.

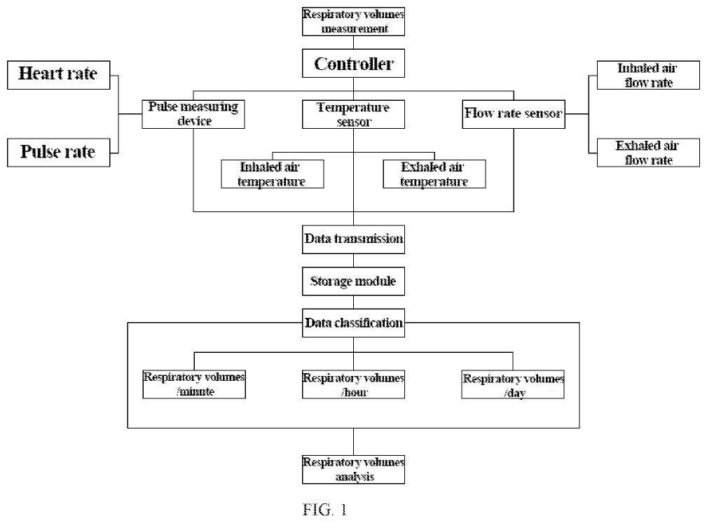

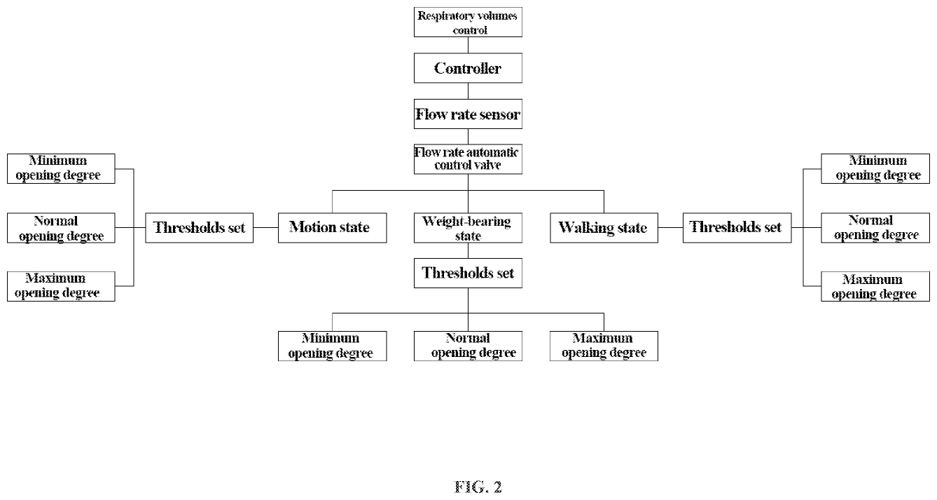

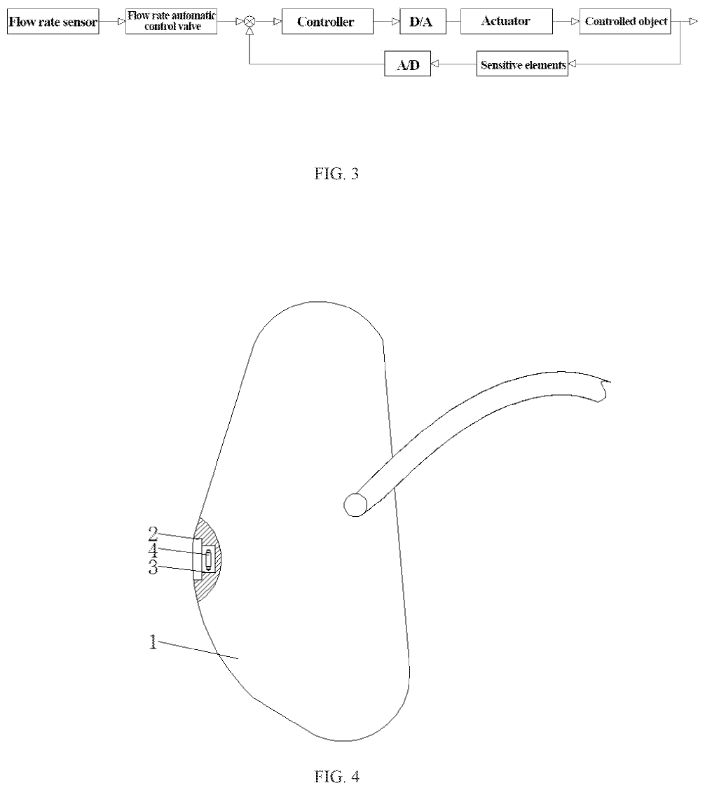

Refer to FIG. 1-4. The real-time sensing measurement for the respiratory volume of the tested person includes a controller, a pulse measuring device, a temperature sensor, a flow rate sensor, and a storage module. The controller receives an electric signal transmitted by the pulse measuring device, the temperature sensor and the flow rate sensor. The controller, the pulse measuring device, the temperature sensor, the flow rate sensor and the storage module communicate through electric signals. The pulse measuring device, the temperature sensor and the flow rate sensor monitor the respiratory volumes of the tested person in real time, and the test data stored in the storage module. Through the cooperation of the pulse measuring device, the temperature sensor, the flow rate sensor and the storage module, the measured respiratory volume is more accurate, and the measured data are stored in the storage module, which is convenient for users to analyze the measured data according to the measured data.

Specifically, the pulse measuring device is configured to measure the heart rate and the pulse rate of the tested person. The temperature sensor is configured to sense an inhaled air temperature and an exhaled air temperature of the tested person. The flow rate sensor is configured to sense an inhaled air flow rate and an exhaled air flow rate of the tested person. The temperature sensor communicates with the flow rate sensor, which makes the temperature sensor and the flow rate sensor calibrate each other through electric signals. Thus the exhaled volumes measurement is conducted in real time, which results more accurate data.

Specifically, the tested person measures the respiratory volumes through the pulse measuring device, the temperature sensor and the flow rate sensor. The measured data is transferred to the storage module. A processor in the storage module classifies the acquired data. The classified data includes the value of the minutely respiratory volumes, an hourly respiratory volumes, and a daily respiratory volumes. The minutely respiratory volumes, the hourly respiratory volumes and the daily respiratory volumes are all stored in the storage module. A dynamic estimation model of the respiratory volume is obtained by analyzing the relationship between the minutely/hourly/daily respiratory volume and the pulse, temperature and flow rate, and the respiratory volume is output, which is convenient for users to grasp their own exhalation volume in real time. Meanwhile, the exhalation volume data is readily shared and provided for professionals to watch, so that professionals can judge whether the exhalation volume of the user is accurate according to the data.

An intelligent control technology apparatus for a respiratory volume of a tested person includes the mask 1 the flow rate sensor 2, the flow rate automatic control valve 3 and the controller 4. The flow rate sensor 2, the flow rate automatic valve 3 and the controller 4 are all installed on the mask 1. The flow rate sensor 2 obtains the respiratory volume inhaled and exhaled by the tested person. Air is exhaled and inhaled through the flow rate automatic control valve 3, and the respiratory volume of each exhalation and inhalation is measured by the flow rate sensor 2. If the respiratory volume of each exhalation and inhalation is higher than a pre-determined threshold value which is stored in the controller and is set according to a maximum respiratory volume in big data, the flow rate sensor 2 will send it to the flow rate automatic control valve 3. Then, the respiratory volume of each exhalation and inhalation will be judged by the controller 4. If the respiratory volume of each exhalation and inhalation is lower or higher than a normal threshold value, the controller 4 will activate an actuator to control sensitive elements inside the flow rate automatic control valve 3 and control an opening size of the flow rate automatic control valve 3. After adjustment, an electric signal will be transmitted to the controller 4 by the flow rate automatic control valve 3, so that the apparatus can be adjusted automatically according to the amount of the exhalation volume by the user.

Specifically, the intelligent control technology apparatus for the respiratory volume of the tested person includes three modes consisting of an exercise state, a weight-bearing state and a walking state. The exercise state, the weight-bearing state and the walking state are respectively set with different thresholds, and the thresholds of the exercise state, the weight-bearing state and the walking state include a minimum opening degree, a normal opening degree and a maximum opening degree. Each mode corresponds to three different opening degrees of a breather valve. The opening degree of the breather valve is minimum when air velocity (±20%) is satisfied with P5 (5th percentile) of breath required for each state; the opening degree of the breather valve is normal when air velocity (±20%) is satisfied with an average amount of breath required for each state; the opening degree of the breather valve is maximum when air velocity (±20%) is satisfied with P95 (95th percentile) of breath required for each state. The flow rate sensor 2, the flow rate automatic control valve 3 and the controller 4 open the maximum opening degree when detecting a large respiratory volume of the tested person, the flow rate sensor 2, the flow rate automatic control valve 3 and the controller 4 open the normal opening degree when detecting a normal respiratory volume of the tested person, and the flow rate sensor 2, the flow rate automatic control valve 3 and the controller 4 open the minimum opening degree when detecting a small respiratory volume of the tested person. According to the user's own condition, different thresholds can be set automatically to make the apparatus more suitable to the user and increase the applicability of the apparatus in use.

It should be noted that in this specification, relational terms such as first and second are only used to distinguish one component or operation from another, and do not necessarily require or imply any such actual relationship or order between these components or operations. Moreover, the terms “include” “comprise” or any other variation thereof are intended to cover nonexclusive inclusion so that a process, method, article, or device includes not only those elements, but also other elements not explicitly listed, or those inherent to such process, method, article or device.

Although the embodiments of the invention have been shown and described, it can be understood that those of ordinary skill in the art can make a variety of changes, modifications, substitutions and modifications to these embodiments without departing from the principle and spirit of the invention, and the scope of the invention is limited by the appended claims and their equivalents.

What is claimed is:

1. A real-time sensing measurement apparatus for a respiratory volume of a tested person, comprising a controller, a pulse measuring device, a temperature sensor, a flow rate sensor and a storage module, wherein
the controller receives electric signals transmitted by the pulse measuring device, the temperature sensor and the flow rate sensor;
the controller, the pulse measuring device, the temperature sensor, the flow rate sensor and the storage module communicate through the electric signals; and
the pulse measuring device, the temperature sensor and the flow rate sensor monitor the respiratory volume of the tested person in real time to obtain test data by the storage module.

2. The real-time sensing measurement apparatus according to claim 1, wherein,
the pulse measuring device is configured to measure a heart rate and a pulse rate;
the temperature sensor is configured to sense an inhaled air temperature and an exhaled air temperature of the tested person;
the flow rate sensor is configured to sense an inhaled air flow rate and an exhaled air flow rate of the tested person; and
the temperature sensor communicates with the flow rate sensor.

3. The real-time sensing measurement apparatus according to claim 1, wherein
the tested person measures the respiratory volume through the pulse measuring device, the temperature sensor and the flow rate sensor, and then acquired data is transferred to the storage module;
a processor in the storage module classifies the acquired data to obtain classified data, wherein, the classified data comprises a minutely respiratory volume, an hourly respiratory volume and a daily respiratory volume, and the minutely respiratory volume, the hourly respiratory volume and the daily respiratory volume are all located in the storage module; and
a dynamic estimation model of the respiratory volume is obtained by analyzing a relationship between the minutely respiratory volume, the hourly respiratory volume, the daily respiratory volume and pulse, temperature and flow rate.

4. An intelligent control technology apparatus for a respiratory volume of a tested person, comprising a mask, a flow rate sensor, a flow rate automatic control valve and a controller, wherein
the flow rate sensor, the flow rate automatic control valve and the controller are all installed on the mask;
the flow rate sensor obtains the respiratory volume inhaled and exhaled by the tested person, air is exhaled and inhaled through the flow rate automatic control valve, and the respiratory volume of each exhalation and inhalation is measured by the flow rate sensor, wherein
if the respiratory volume of the each exhalation and inhalation is higher than a pre-determined threshold value, the flow rate sensor sends the respiratory volume of the each exhalation and inhalation to the flow rate automatic control valve;
the respiratory volume of the each exhalation and inhalation is judged by the controller, wherein
if the respiratory volume of the each exhalation and inhalation is lower or higher than a normal threshold value, the controller activates an actuator to control sensitive elements inside the flow rate automatic control valve and control an opening size of the flow rate automatic control valve; and
after adjustment, an electric signal is transmitted to the controller by the flow rate automatic control valve.

5. The intelligent control technology apparatus according to claim 4, wherein the intelligent control technology apparatus comprises three modes consisting of an exercise state, a weight-bearing state and a walking state, wherein
the exercise state, the weight-bearing state and the walking state are respectively set with different thresholds, and the thresholds of the three modes comprise a minimum opening degree, a normal opening degree and a maximum opening degree, wherein
the flow rate sensor, the flow rate automatic control valve and the controller open the maximum opening degree when detecting a large respiratory volume of the tested person;
the flow rate sensor, the flow rate automatic control valve and the controller open the normal opening degree when detecting a normal respiratory volume of the tested person; and
the flow rate sensor, the flow rate automatic control valve and the controller open the minimum opening degree when detecting a small respiratory volume of the tested person.
